(19) United States
(12) Patent Application Publication
(10) Pub. No.: US 2005/0170013 A1
(43) Pub. Date: Aug. 4, 2005

(54) Composition for treating a dermatological inflammatory response and method of preparation thereof

(76) Inventor: Jerry A. Douglas, Raleigh, IL (US)

Correspondence Address: Douglas W. Schelling, Ph.D., 414 Union Street, Suite 2020, Bank of America Plaza, Nashville, TN, 37219, US

(21) Appl. No.: 10/771,928

(22) Filed: Feb. 4, 2004

(51) Int. Cl. A61K 33/40; A61K 33/32

(52) U.S. Cl. 424/616; 424/641; 514/574; 514/566

(57) ABSTRACT

The present invention discloses a composition of matter having anti-inflammatory characteristics to be used in the treatment of dermatological inflammatory responses. The composition includes four salts and an oxidant. Also disclosed herein are methods of preparing the composition and methods of using the compositions.

### BACKGROUND OF THE INVENTION

Hydrogen peroxide and peroxide-containing preparations are known to be effective therapeutic and prophylactic treatments for conditions involving inflammation, such as periodontal hemorrhaging, gingivitis, periodontitis and other oral conditions. Frazier et al., U.S. Pat. No. 4,980,152, disclose an oral preparation containing between 0.5% and 10% hydrogen peroxide. Schiraldi et al., U.S. Pat. No. 4,992,259, disclose the use of zinc compounds including zinc chloride as astringent-desensitizing agents. Additional hydrogen peroxide containing preparations are disclosed in U.S. Pat. No. 5,104,644, issued to Douglas on Apr. 14, 1992; U.S. Pat. No. 5,174,990, issued to Douglas on Dec. 29, 1992; and U.S. Pat. No. 5,310,546, issued to Douglas on May 10, 1994, all of which are hereby incorporated by reference in their entirety.

Preparations containing other ingredients in conjunction with hydrogen peroxide, however, have generally been unstable in storage; as the hydrogen peroxide reacts with one or more of the other ingredients in the preparation and components of the preparation tend to precipitate. As a result, the capacity of the hydrogen peroxide to release oxygen and of the other ingredients to perform their respective functions in a given preparation is lost or greatly diminished even after relatively short storage periods as, for example, on a drug store display shelf. Additionally, the instability of preparations, such as mouth rinses, containing hydrogen peroxide has been known to render them unsatisfactory with respect to shelf-life requirements of the United States Food and Drug Administration.

### SUMMARY OF THE INVENTION

The present invention discloses a composition of matter, which reduces or blocks a dermatological inflammatory response. The composition includes four salts and an oxidant. The composition of the present invention is prepared by the method disclosed herein. The method of preparation includes incubation steps, which are critical. Also disclosed herein are methods of using the composition of the present invention to treat or prevent the dermal inflammatory responses associated with insect bites, burns, diaper rash, jock itch, and other irritants.

The present invention, a composition for treating a dermatological inflammatory response, includes from about 0.02% to about 0.08% disodium EDTA, from about 0.04% to about 0.20% sodium lauryl sulfate, from about 0.015% to about 0.20% sodium citrate, from about 0.01% to about 0.019% zinc chloride, and from about 0.5% to about 3% of an oxidant. In certain embodiments, the composition such that the ETDA, the sodium lauryl sulfate, and the sodium citrate are mixed in an aqueous solution in an acidic pH range of from about 3.5 to about 4.5. In other embodiments, the amount of citric acid is from about 0.01% to about 0.02%. Other embodiments include from about 3% to about 4% glycerin, about 3.6% to about 4.0% glycerin, or about 3.64% glycerin. The oxidant may be hydrogen peroxide, or 3% hydrogen peroxide.

The present invention, a composition for treating a dermatological inflammatory response, includes from about 0.05% to about 0.06% disodium EDTA, from about 0.07% to about 0.08% sodium lauryl sulfate, from about 0.015% to about 0.80% zinc chloride, from about 0.02% to about 0.03% sodium citrate, and from about 1.8% to about 2.1% of an oxidant. The composition should be mixed so that the ETDA, the sodium lauryl sulfate, and the sodium citrate are mixed in an aqueous solution in an acidic pH range of from about 3.5 to about 4.5. The oxidant can be, for illustration, but not limitation, hydrogen peroxide. In certain embodiments, the zinc chloride is present in an amount from about 0.01% to about 0.02%. The composition may also include from about 0.01% to about 0.02% citric acid. Alternately, the present invention includes a composition for treating a dermatological inflammatory response having about 0.053% disodium EDTA, about 0.077% sodium lauryl sulfate, about 0.019% zinc chloride, about 0.029% sodium citrate, and about 1.9% of an oxidant.

The method of preparing the composition for treating a dermatological inflammatory response, disclosed herein, includes dissolving disodium EDTA in deionized water to prepare a solution, adding sodium lauryl sulfate to the solution, adding sodium citrate to the solution, incubating the solution for about 5-10 minutes so that heat dissipates, adding zinc chloride to the solution, incubating the solution for about 5-10 minutes so that heat dissipates, mixing the solution for about 30-60 minutes, incubating the solution for a minimum of 4 hours, adding an oxidant to the solution, and mixing the solution for about 30-45 minutes. In certain embodiments, the oxidant is hydrogen peroxide. The present invention also includes a composition for treating a dermatological inflammatory response as prepared in this paragraph, wherein the composition includes about 0.053% disodium EDTA, about 0.077% sodium lauryl sulfate, about 0.019% zinc chloride, about 0.029% sodium citrate, and about 1.9% oxidant.

The present invention also includes a method of treating a dermatological inflammatory response, which includes providing a composition having: from about 0.05% to about 0.06% disodium EDTA, from about 0.07% to about 0.08% sodium lauryl sulfate, from about 0.01% to about 0.02% zinc chloride, from about 0.02% to about 0.03% sodium citrate, from about 1.8% to about 2.1% oxidant, and applying the composition to an area of a body having the dermatological inflammatory response. Certain embodiments of the invention include applying the composition at least one time daily, at a frequency of from 3 to 5 times per day, or while the dermatological inflammatory response is present. Other embodiments of the invention include applying the composition to an exposed skin surface, such as an arm, or a non-exposed skin surface, such as a foot or genital area.

Accordingly, it is an object of this invention to provide a chemically stable preparation containing an amount of hydrogen peroxide that is effective for therapeutic and preventative treatment of a dermatological inflammatory response.

It is another object of the present invention to provide a composition that efficiently reduces or eliminates dermal inflammation by the formulation being able to impede, alter or block the biochemical process associated with the tissue reaction initiated by exogenous stimuli.

Still another object of the present invention is to provide a composition that blocks the inflammation chemical and biochemical pathway that leads to a dermatological inflammatory tissue reaction.

Yet another object of the present invention is to provide a method of preparation for a composition that reduces or prevents a dermatological inflammatory response.

### DETAILED DESCRIPTION OF THE PREFERRED EMBODIMENTS

The present invention discloses a composition of matter to treat a dermatological inflammatory response. The composition includes disodium EDTA, sodium lauryl sulfate, sodium citrate, zinc chloride, and an oxidant. The invention also discloses a method of preparing the above-referenced composition. Finally, the invention discloses methods of using the composition.

The composition of the present invention is a unique blend of chemicals with specific concentrations that allow the four ions that disassociate when the cations and anions are mixed in an aqueous solution in an acidic range of 3.5 to 4.5. Such mixture allows the interaction of the four ions to form a physiological solution when complexed with an oxidant that reduces or stops an inflammatory reaction and its side effects. Such an inflammatory reaction may be initiated by an exogenous stimuli, for example, an insect bite. The composition gives relief to the sting, itching and edema caused by the tissue reaction to an exogenous stimuli, or other non-exogenous dermal inflammation, such as certain types of lupus, psoriasis, genital rash, hives, viral skin rash, and other systemic origin dermatological reactions.

When a dermal surface is subjected to exogenous stimuli, such as virus, fungi or insect bite, the exposure can provoke a complex reaction in the vascularized connective tissue of the skin surface. This inflammatory reaction will progress to various stages with discomfort and tissue destruction unless the noxious stimuli are neutralized. The vascular and cellular responses of both acute and chronic inflammation are mediated by chemical factors. These inflammatory mediators working individually or in combination can increase and amplify the inflammatory response and influence its evolution. Such an inflammatory response can initiate cell necrosis or tissue damage that can compound and increase the triggering of more inflammatory mediators. Generally, when exogenous stimuli are neutralized and the inflammatory mediators are blocked or inhibited, the tissue may begin to repair.

The composition of the present invention, which includes four salts and an oxidant, is believed by the inventor to have ability to block the chemical inflammatory mediators such as eicosanoids, arachidonic acid, histamine, bradykinin, leukotrienes, substance P. and many other classes of chemical mediators that form in response to exogenous stimuli.

Specifically, arachidonic acid metabolites can mediate every phase of the inflammatory process. Furthermore, products derived from the metabolism of arachidonic acid have a diverse affect on many biological processes that occur in the inflammatory process. Briefly, arachidonic acid, a 20-carbon polyunsaturated fatty acid, is normally esterified in membrane phospholipids. Typically, such esterification occurs in the carbon 2 position of phosphatidyl choline, phosphatidylino-sitol and phospholipids ethanolamine. Arachidonic acid is released from the membrane due to a physical, mechanical or chemical stimulus, such as an insect bite. This invention interferes with this process due to the zinc ion, complexed by the 3 other ions and the oxidant, ability to bind to one of the substrates produces in this complex biochemical process, changing the electrical field from a neutral base to a positive charge.

Pursuant to the present invention, as further described below and without being bound by mechanism or theory, when the chemically complexed zinc ion is mixed with sodium and citrate ions, the resulting composition is believed by the inventor to have the ability to penetrate a cell membrane and alter the metabolic function of the cell in order to inhibit or block the production of the arachidonic acid metabolites that are responsible for initiating and exacerbating the inflammatory process.

The composition of the present invention is believed by the inventor to affect the synthesis of two major classes enzymes, cyclooxygenases and lipooxygenases. Specifically, due to the high affinity between zinc and oxygen [ZH═ZnOz ⁻²Zn(OH)z⇄Zn⁺²+Z(OH)—], the complexed zinc ion binds to the oxygen atom which changes the group from a nucleophile to a positive charged functional group, which suppresses enzyme production. The suppression of cyclooxygenase production helps inhibit the inflammatory process. Furthermore, without being bound by mechanism or theory, the zinc chloride and sodium citrate complexed with an oxidant reduce or block the production of inflammatory mediators by any leukocytes that leak from the vascular system which form the microvascular complex in the area of the tissue that has reacted to an exogenous stimulus.

Another function of the present invention is believed by the inventor to be its ability to change the function, chemical composition and action of the proteolytic enzyme elastase (an inflammatory mediator), by changing the enzymatic functional groups from nucleophilic, electron donating, to electrophilic, electron-accepting group, by the binding of the zinc ion to one of the chemical substrates produced in the inflammatory process. Without being bound by mechanism or theory, the zinc binds the substrates produced in the elastase enzymatic functional groups. For example, the positively charged zinc ion forms a complex with the hydroxide ion of a substrate, which alters its chemical function. The hydrolysis of an ester or an amide during the function of enzymatic activity of elastase, the inflammatory mediator, is to replace water by a stronger nucleophilic group that is part of the enzyme's active site. The two-step pathway that occurs in this process requires that the intermediate be more susceptible to nucleophilic attack by water than the original ester or amide. Nucleophilic groups on enzymes participate in a variety of other types of reactions in addition to hydrolytic reactions. By changing the nucleophilic group to an electrophilic group, production of the inflammatory mediators produced by elastase is impeded.

It is believed by the inventor that the positive charged zinc ion, when complexed with the other three salts and oxidant, forms complexes with the carbonyl oxygen atom of the aldehyde or peptide substrate when the zinc ion is present at an active inflammatory site, changing the field from a neutral to a positive charged chemical field. Such binding interferes with the chemical process of the inflammatory mediators, such as the prostaglandin group that includes a number of double-bond compounds that are affected by the charge of the electrical field. Some of these compounds have restricted tissue distribution, and changing the electrical charge impedes the tissue distribution more. The impediment of this process helps reduce the effects of the dermal inflammatory process, pain and increased temperature (fever) and edema in the area affected by the exogenous stimuli.

This chemical alteration of the inflammatory mediators interferes with alterations in the vascular complex that increase blood flow to an inflamed area. Further, the altered inflammatory mediators block the structural change in the microvasculature in the skin, which would normally permit plasma proteins and leukocytes to leave the circulatory system, which causes the immigration of the leukocytes from the altered microvasculature complex to the exogenously stimulated area. Those leukocytes produce eicosanoids, which generate a low protein fluid that results in edema in the exogenously stimulated area. Stated another way, by preventing the production of functional inflammatory mediators by the leukocytes, the process of inflammation is reduced or blocked.

Zinc chloride is a component of the composition. Although zinc chloride advantageously has the ability to interfere with metabolic activity of pathogenic microbiota, it also helps to reduce inflammation and restore edematous tissue to a normal state. In certain embodiments, the composition comprises between about 0.005% and about 0.1% zinc chloride (all percentages herein are on a weight/volume basis unless indicated otherwise). Zinc chloride in amounts significantly less than about 0.005% would be insufficient to provide the desired therapeutic effect. In alternate embodiments, the amount of zinc chloride is from about 0.015% to about 0.80%. In still other embodiments, the composition has from about 0.01% zinc chloride to about 0.02% zinc chloride. In other embodiments, the amount of zinc chloride is from about 0.01% to about 0.019%. In other embodiments, the amount of zinc chloride is from about 0.02% to about 0.05%. In yet other embodiments, the composition has from about 0.05% zinc chloride to about 0.80% zinc chloride. Other embodiments have 0.019% zinc chloride.

The composition includes sodium lauryl sulfate as a surfactant. Without being bound to any theory, it is believed by the inventor that sodium lauryl sulfate serves to enhance the interaction among the component and tissue, or skin. Additionally, sodium lauryl sulfate makes cell walls more permeable and enhances the ability of zinc chloride to perform its described function. In certain embodiments, the composition contains an amount of sodium lauryl sulfate from about 0.04% to about 0.20%. In other embodiments, the amount of sodium lauryl sulfate in the composition is from about 0.07% to about 0.08%. In still other embodiments, the composition contains an amount of sodium lauryl sulfate from about 0.04% to about 0.07%. In yet other embodiments, the amount of sodium lauryl sulfate in the composition is from about 0.20% to about 0.08%. Other embodiments have 0.077% sodium lauryl sulfate.

An anticoagulant to aid in the healing hemorrhaged tissue is also provided in the composition. The preferred anticoagulant is sodium citrate, which also serves as an antimicrobial enhancer and an anti-inflammatory agent when complexed with a heavy metal ion such as zinc. Sodium citrate is provided in an amount of from about 0.05% to about 0.20%. In other embodiments, sodium citrate is present in an amount from about 0.02% to about 0.03%. In other embodiments, 0.029% sodium citrate is present.

The composition of the present invention comprises an oxygen-releasing agent, which also acts as an astringent and anti-inflammatory agent, specifically, 3% hydrogen peroxide. The composition includes between about 0.5% and about 3.0% of an oxidant, such as hydrogen peroxide (all percentages herein are on a weight/volume basis unless indicated otherwise). In certain embodiments, hydrogen peroxide is present in an amount of from about 1.8% to about 2.1%. In still other embodiments, hydrogen peroxide is present in an amount of about 1.9%. Hydrogen peroxide in amounts significantly less than about 0.25% would be insufficient to provide the desired therapeutic effect, whereas amounts significantly greater than about 3.0% would be potentially unstable under conditions of prolonged storage. The present invention provides a composition of matter that is stable. Stability tests using time, temperature and atmospheric pressure show the formulation remains stable, maintaining the hydrogen peroxide and zinc concentrations up to three years. Bench tests of direct sunlight for extended periods of time (10 hours per day for 5 days) also did not alter the concentrations of hydrogen peroxide and zinc.

Citric acid is provided in the composition for purposes of demineralization and stabilization. Citric acid is effective for adjusting and maintaining the pH of the composition in a range at which the hydrogen peroxide remains stable, roughly from about 3.5 to about 4.5. The citric acid content, which is substantially always greater than about 0.005%, is therefore that amount which is effective for achievement of the desired pH. In certain embodiments, citric acid is present in an amount from about 0.01% to about 0.02%.

Any of a variety of pharmaceutically acceptable carrier media may be used including an aqueous alcohol. The presence of alcohol in the carrier provides sterilization capacity and is thought to influence product stability. The amount of alcohol present in the composition is from about 0.5% to about 1.6%. In other embodiments, the amount of alcohol present is about 3.0%. In still other embodiments, the amount of alcohol present from about 1.7% to about 3.0%.

The alcohol may be denatured with any of a variety of denaturing agents, alone or in combination with, including, nonexclusively, anethole, anise oil, bay oil, bergamot oil, bitter almond oil, cedar leaf oil, cinnamic aldehyde, cinnamon oil, clove oil, eucalyptol, eucalyptus oil, eugenol, lavender oil, menthol, peppermint oil, sassafras oil, spearmint oil, terpeneless spearmint oil, thyme oil, thymol and/or wintergreen oil. Generally, less than about 0.1% total denaturing agent is preferred in the composition. In certain embodiments, the alcohol also contains poloxamer 407, or another solubilizer, to solubilize the denaturing agents. Generally, less than about 1% poloxamer 407 is present.

In addition to the above-described components, the composition of the present invention may also contain glycerin, which is believed to serve as an additional surfactant. Glycerin may be present in the range of from about 1.8% to about 9.0%. In other embodiments, glycerin may be present in an amount from about 3.0% to about 4.0%. In still other embodiments, glycerin may be present in an amount from about 3.6% to about 4.0%.

The composition of the present invention also contains an appropriate chelating agent to keep the various minerals in combination. The preferred chelating agent is disodium EDTA provided in the range of from about 0.02% to about 0.08%. Alternately, the amount of disodium EDTA in the composition is from about 0.05% to about 0.06%. In other embodiments, the amount of disodium EDTA in the composition is from about 0.02% to about 0.05%. In still other embodiments, the amount of disodium EDTA in the composition is from about 0.06% to about 0.08%. In yet other embodiments, the amount of disodium EDTA in the composition is about 0.053%.

Any of a number of pharmaceutically safe and compatible coloring agents, including, but not limited to, D & C yellow #10 and D & C green #3, may also be used in effective amounts to enhance the composition.

Method of Preparation

Disclosed herein is a method of preparing the composition of the present invention. Each of the elements of the composition is well known in the art and is commercially available from multiple sources. For example, an alcohol solution comprising ethanol, denaturing agents and a solubilizer is provided herein. This alcohol solution may be prepared by obtaining the elements from a commercial source and mixing its components until clear.

By following the method of preparation disclosed herein, the mixture of the four salts and oxidant in an aqueous solution allows the ions to separate and interact with each other.

A solution having disodium EDTA, sodium lauryl sulfate, sodium saccharin, sodium citrate and other discretionary additives, such as glycerin, is prepared by mixing the above referenced components with de-ionized water. In certain embodiments, the method includes dissolving disodium EDTA in deionized water to prepare a solution, adding sodium lauryl sulfate to the solution, adding sodium citrate to the solution, and incubating the solution for about 5-10 minutes so that heat dissipates.

After incubating the solution, citric acid and zinc chloride are added to the solution. After the addition of zinc chloride, the solution is incubated for about 5-10 minutes so that heat dissipates. In certain embodiments, an alcohol pre-mix, including alcohol SB38, menthol, peppermint oil, poloxamer 407, is mixed into the solution. The alcohol solution is mixed with the solution having the zinc chloride and the remaining ingredients until it is substantially clear. To achieve a composition having stabilized hydrogen peroxide, the heat generated from this mixing process is allowed to dissipate prior to further processing. For example, the solution is mixed for 30-60 minutes and incubated, or allowed to sit while not mixing, for a minimum of 4 hours. After incubation, an oxidant, for example hydrogen peroxide, is added to the solution and solution is mixed for about 30-45 minutes.

When adding zinc chloride to the solution, the zinc chloride is dissolved in the solution and mixed until the solution is clear. Again, it is important to note that to achieve a composition having stabilized hydrogen peroxide, the heat generated from this mixing process is allowed to dissipate prior to further processing of the solution now containing zinc chloride.

The composition of the present invention that is prepared in accordance with the method of this invention has superior chemical stability and, consequently, shelf life. The composition is stable, i.e., they remain clear, with no visible formation of precipitates or detectable evolution of gases after prolonged periods of storage.

Method of Use of Composition

The composition disclosed herein can be used in a variety of methods to treat dermal inflammation. The composition can be applied to inflamed skin from about 3 to about 5 times per day. To do so, a generous application of the composition is topically applied to the skin and allowed to absorb into and/or dry on the skin, as ordinarily done with any dermatologic ointment. In some situations, the composition is applied to the affected skin at least one time daily. In still other situations, the composition is applied frequently while a dermatological inflammatory response is present.

The following examples illustrate the invention.

### EXAMPLE I

A composition for treating a dermal inflammation event is prepared according to this example. The following components are added to 94.64 liters of deionized water: 49.90 grams of disodium EDTA, 72.58 grams of sodium lauryl sulfate, 27.22 grams of sodium saccharin, 27.22 grams of sodium citrate and 3442.82 grams of 99% glycerin. The solution is mixed until all components are dissolved and mixing continues so that the heat dissipates, about 5-10 minutes.

The pH of the solution is confirmed to be in the range of 3.5-4.5.

18.14 grams of citric acid and 18.14 grams of zinc chloride are added to the above referenced solution. Again, the solution is mixed until all components are dissolved and mixing continues so that the heat dissipates, about 5-10 minutes.

3129.84 grams of an alcohol pre-mix are added to the solution. The alcohol pre-mix includes 439.99 grams of pluronic F-127. The solution is mixed until all components are dissolved and mixing continues so that the heat dissipates, about 5-10 minutes. The solution is then mixed for an additional 30 minutes and allowed to stand for 5 hours. The pH is measured and confirmed to be in the range of 3.5-4.5. To the solution are added 24.84 liters of 3% hydrogen peroxide. The solution is mixed for 30 minutes.

### EXAMPLE 2

A composition having the following components is prepared by the method as set forth in example I.

| Component | % (weight/volume) |
|---|---|
| Glycerin | 3.64 |
| Sodium lauryl | 0.077 |
| Sulfate | |
| Disodium EDTA | 0.053 |
| Alcohol pre-mix | 3.30 |
| (including Pluronic F-127 0.465%) | |
| Sodium citrate | 0.029 |
| Sodium saccharin | 0.029 |
| Zinc chloride | 0.019 |
| Hydrogen peroxide | 1.92 |
| Citric acid | 0.019 |

This patent application incorporates by reference all references and publications disclosed herein.

Thus, although there have been described particular embodiments of the present invention of new and useful Composition for Treating a Dermatological Inflammatory Response and Method of Preparation Thereof, it is not intended that such references be construed as limitations upon the scope of this invention except as set forth in the following claims.

1. A composition for treating a dermatological inflammatory response, comprising:
from about 0.02% to about 0.08% disodium EDTA;
from about 0.04% to about 0.20% sodium lauryl sulfate;
from about 0.015% to about 0.20% sodium citrate;
from about 0.01% to about 0.019% zinc chloride; and
from about 0.5% to about 3% oxidant.

2. The composition of claim 1, wherein the ETDA, the sodium lauryl sulfate, and the sodium citrate are mixed in an aqueous solution in an acidic pH range of from about 3.5 to about 4.5.

3. The composition of claim 1, further comprising from about 0.01% to about 0.02% citric acid.

4. The composition of claim 1, further comprising from about 3.6% to about 4.0% glycerin.

5. The composition of claim 1, wherein the oxidant is hydrogen peroxide.

6. The composition of claim 5, wherein the oxidant is 3% hydrogen peroxide.

7. A composition for treating a dermatological inflammatory response, comprising:
from about 0.05% to about 0.06% disodium EDTA;
from about 0.07% to about 0.08% sodium lauryl sulfate;
from about 0.015% to about 0.80% zinc chloride;
from about 0.02% to about 0.03% sodium citrate; and
from about 1.8% to about 2.1% oxidant.

8. The composition of claim 7, wherein the ETDA, the sodium lauryl sulfate, and the sodium citrate are mixed in an aqueous solution in an acidic pH range of from about 3.5 to about 4.5.

9. The composition of claim 8, wherein the oxidant is hydrogen peroxide.

10. The composition of claim 9, wherein the zinc chloride further comprises from about 0.01% to about 0.02% zinc chloride.

11. The composition of claim 7, further comprising from about 0.01% to about 0.02% citric acid.

12. A method of preparing a composition for treating a dermatological inflammatory response, comprising:
dissolving disodium EDTA in deionized water to prepare a solution;
adding sodium lauryl sulfate and sodium citrate to the solution;
incubating the solution for about 5-10 minutes so that heat dissipates;
adding zinc chloride to the solution;
incubating the solution for about 5-10 minutes so that heat dissipates;
mixing the solution for about 30-60 minutes;
incubating the solution for a minimum of 4 hours;
adding an oxidant to the solution; and
mixing the solution for about 30-45 minutes.

13. The method of claim 12, wherein adding an oxidant further comprises adding hydrogen peroxide.

14. A composition for treating a dermatological inflammatory response as prepared in claim 13, wherein the composition further comprises,
about 0.053% disodium EDTA;
about 0.077% sodium lauryl sulfate;
about 0.019% zinc chloride;
about 0.029% sodium citrate; and
about 1.9% oxidant.

15. A method of treating a dermatological inflammatory response, comprising:
providing a composition having:
from about 0.05% to about 0.06% disodium EDTA;
from about 0.07% to about 0.08% sodium lauryl sulfate;
from about 0.01% to about 0.02% zinc chloride;
from about 0.02% to about 0.03% sodium citrate;
from about 1.8% to about 2.1% oxidant; and
applying the composition to an area of a body having the dermatological inflammatory response.

16. The method of claim 15, wherein applying the composition further comprises applying the composition at least one time daily.

17. The method of claim 15, wherein applying the composition further comprises applying the composition at a frequency of from 3 to 5 times per day.

18. The method of claim 17, wherein applying the composition further comprises applying the composition while the dermatological inflammatory response is present.

19. The method of claim 15, wherein applying the composition further comprises applying the composition to a skin surface.

20. A composition for treating a dermatological inflammatory response, comprising:
about 0.053% disodium EDTA;
about 0.077% sodium lauryl sulfate;
about 0.019% zinc chloride;
about 0.029% sodium citrate; and
about 1.9% oxidant.
